(19) United States
(12) Patent Application Publication
(10) Pub. No.: US 2024/0285605 A1
(43) Pub. Date: Aug. 29, 2024

(54) DRIED COSMETIC PREPARATION FOR REHYDRATION AND USE

(71) Applicant: Onelogy Inc., Brooklyn, NY (US)

(72) Inventors: Roxana Estelle Bazgoneh, Brooklyn, NY (US); Krupa Koestline, Orlando, FL (US); Mathias Letan Martin, Robion (FR); Laura Sitruk, Salon-de-Provence (FR); Mélanie LLOBELL Canetto, Eyguières (FR)

(73) Assignee: Onelogy Inc., Brooklyn, NY (US)

(21) Appl. No.: 18/590,056

(22) Filed: Feb. 28, 2024

Related U.S. Application Data
(60) Provisional application No. 63/487,420, filed on Feb. 28, 2023.

(51) Int. Cl. A61K 31/455; A61K 8/34; A61K 8/365; A61K 8/37; A61K 8/44; A61K 8/49; A61K 8/60; A61K 8/64; A61K 8/67; A61K 8/73; A61K 8/9794; A61K 9/00; A61K 9/19; A61K 31/198; A61K 31/215; A61K 31/355; A61K 31/366; A61K 31/7032; A61K 31/7034; A61K 31/721; A61K 36/886; A61K 38/06; A61K 38/08; A61K 47/10; A61K 47/12; A61K 47/36; A61Q 19/00

(52) U.S. Cl. CPC A61K 31/455; A61K 8/345; A61K 8/365; A61K 8/37; A61K 8/44; A61K 8/498; A61K 8/602; A61K 8/64; A61K 8/675; A61K 8/678; A61K 8/73; A61K 8/733; A61K 8/9794; A61K 9/0014; A61K 9/19; A61K 31/198; A61K 31/215; A61K 31/355; A61K 31/366; A61K 31/7032; A61K 31/7034; A61K 31/721; A61K 36/886; A61K 38/06; A61K 38/08; A61K 47/10; A61K 47/12; A61K 47/36; A61Q 19/00; A61K 2800/10; A61K 2800/31; A61K 2800/84

(57) ABSTRACT

One or more techniques, systems and articles of manufacture are described for a product that is dried and can subsequently be rehydrated to produce a cosmetic or pharmaceutical. A molded and dried preparation has water weight of greater than zero and less than 50 percent. The dried preparation is form by drying a liquid preparation that contains a base material, an active ingredient and an inactive ingredient. The molded and dried preparation is configured to be rehydrated with water or another aqueous based solution or emulsion. The preparation may rapidly dissolve with the solvent to obtain a ready-to-use liquid or semi-solid product for topical cosmetic or pharmaceutical application.

### RELATED APPLICATIONS

This application claims priority to U.S. Ser. No. 63/487,420, entitled DRIED COSMETIC PREPARATION FOR REHYDRATION AND USE, filed Feb. 28, 2023, all of which is incorporated herein by reference.

### BACKGROUND

Cosmetics and pharmaceuticals are commonly used in everyday routines. Travel and general daily activities, like going to the gym or work, can require having access to cosmetics and throughout the day or week to maintain a cosmetics routine, like a skincare routine. Carrying the cosmetics and pharmaceuticals while traveling or otherwise proceeding through the day may be cumbersome to the user. Further, most active ingredients in liquid products lose up to 50% efficacy in as little as 60 days. This may lead to increased waste as cosmetic and pharmaceutical products may be discarded and replaced frequently because of this efficacy loss.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

In one implementation, a molded article may contain one or more active substances and one or more non-active substances. The molded article may be configured to be rehydrated with water or another aqueous based solution or emulsion. The molded article may be configured to rapidly dissolve or at least move into a mixture, suspension or the like, with the solvent to obtain a ready-to-use liquid or semi-solid product for topical cosmetic or pharmaceutical application. The molded article may be formed by a removal of water from the base formula, such as by a lyophilization, or freeze drying, or other forms of drying, process. For example, freeze drying can comprise a plurality of freezing steps, a plurality of primary drying steps, and at least one secondary drying step. In this example, the plurality of freezing steps may include a ramp sub-step where the temperature gradually decreases over a set time interval followed by a holding sub-step where the target temperature is held for a set time interval. Similarly, the plurality of primary drying steps and at least one secondary drying steps may include a ramp sub-step where the temperature gradually increases over a set time interval followed by a holding sub-step where the target temperature is held for a set time interval. The plurality of primary drying steps and at least one secondary drying steps may be conducted under vacuum pressure.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

What is disclosed herein may take physical form in certain parts and arrangement of parts, and will be described in detail in this specification and illustrated in the accompanying drawings which form a part hereof and wherein:

FIG. 1 is a process flow of a first embodiment of a manufacturing method of freeze-dried cosmetic tablet disclosed herein.

FIG. 2 is a process flow of a second embodiment of a manufacturing method of freeze-dried cosmetic tablet disclosed herein.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

The disclosed molded and dried articles may provide cosmetics and pharmaceuticals in an exact dose of active ingredients that are capable of compact handling and travel. After drying, the articles may contain a residual moisture of 0 to 50%. For example, the articles may contain a residual moisture of >1 to 40%; or 5-30%; 5-20%; 5-15%; 5-10%; or less than 10% by weight For use as a topical cosmetic and pharmaceutical, the articles may be rapidly dissolved in water or another aqueous base solution or emulsion to obtain a ready-to-use liquid or semi-solid end product. The articles may dissolve in a range of 1 second to 60 minutes. After rehydration and dissolution, the end product may have a ph value of less than nine. The end product, a dissolvable tablet, delivers peak potency with minimum waste when compared to traditional bottled products

The rapidly dissolving articles may contain at least one active ingredient and may contain an inactive ingredient. Prior to drying (e.g., lyophilization), the at least one active ingredient and the inactive may be in a liquid preparation, like an aqueous based solution or suspension. The liquid preparation may use, individually or in combination with each other, a base like alginate, chitosan, collagen, sodium hyaluronate (Hyaluronic Acid), Trehalose or other excipients. To form the rapidly dissolving article, the liquid preparation may be freeze-dried. In its dried (e.g., freeze-dried) form, the rapidly dissolving article may contain a single dose of the at least one active ingredient. The dried article can be dissolved in water or another aqueous base solution or emulsion to rehydrate the active and inactive ingredients. Once rehydrated, the end product may be a ready-to-use liquid or semi-solid topical cosmetic or pharmaceutical containing the single dose of the at least one active ingredient. Once rehydrated, the end product may provide single dose of the active ingredient meaning a single application or use of the end product cosmetic or pharmaceutical. Alternatively, the end product may provide a plurality of single doses of the active ingredient. The end product, once rehydrated, may provide multiple application or uses of the preparation as a cosmetic or pharmaceutical that may be stored in a container.

Exemplary active ingredients will be discussed in the following paragraphs. Before being dissolved into the liquid preparation, the active ingredients or their derivatives may be in a pure, blended, liposomal, or hydrolyzed form. A nonexhaustive list of the types of active ingredients includes: amino acids, enzymes and coenzymes, sugars, saccharides (including, but not limited to, polysaccharides, monosaccharides, disaccharides, and oligosaccharides), glycols, polyols, nutrients, phenols, plant extracts, proteins, UV filters, vitamins, organic compounds, inorganic compounds, antioxidants, prebiotics, probiotics, postbiotics, fatty acids, glycerol, triglycerides, monoglycerides, diglycerides, phospholipids, steroids, sterols, sphingolipids, and oils. It will be appreciated that one skilled in the art may recognize that other types of active ingredients may be used in the liquid preparation. The below listed minimum and maximum ranges may be the percentage amount of the ingredients in the liquid preparation if that ingredient is used in the formulation of the cosmetic or pharmaceutical. It will be appreciated while the below tables group ingredients together, many of these ingredients may fit into multiple of the below types of active ingredients.

The at least one active ingredient may be an alpha hydroxy acid (AHA), a beta hydroxy acid (BHA), or a polyhydroxy acid (PHA). These ingredients may be included in the formulation of the liquid preparation as a chemical exfoliant. The table below is a nonexhaustive list of AHAs, BHAs, and PHAs.

| | | Min. | Max. |
|---|---|---|---|
| Ingredient | Classification | Range (%) | Range (%) |
| Citric acid | Alpha hydroxy acid | 0.001 | 100 |
| Glycolic acid | Alpha hydroxy acid | 0.001 | 100 |
| Lactic acid | Alpha hydroxy acid | 0.001 | 100 |
| Malic acid | Alpha hydroxy acid | 0.001 | 100 |
| Mandelic acid | Alpha hydroxy acid | 0.001 | 100 |
| Phytic acid | Alpha hydroxy acid | 0.001 | 100 |
| Tartaric acid | Alpha hydroxy acid | 0.001 | 100 |
| Betaine salicylate | Beta hydroxy acid | 0.001 | 100 |
| Salicylic acid | Beta hydroxy acid | 0.001 | 100 |
| Willow bark extract | Beta hydroxy acid | 0.001 | 100 |
| Gluconolactone | Polyhydroxy acid | 0.001 | 100 |
| Lactobionic acid | Polyhydroxy acid | 0.001 | 100 |
| Maltobionic acid | Polyhydroxy acid | 0.001 | 100 |
| Phytic acid | Polyhydroxy acid | 0.001 | 100 |

The at least one active ingredient may also be an amino acid. The table below is a nonexhaustive list of amino acids that may be used in the rapidly dissolving article.

| | | Min. | Max. |
|---|---|---|---|
| Ingredient | Classification | Range (%) | Range (%) |
| Acetyl glycyl beta-alanine | Amino acid | 0.001 | 100 |
| L-arginine | Amino acid | 0.001 | 100 |
| Asparagine | Amino acid | 0.001 | 100 |
| Aspartic acid | Amino acid | 0.001 | 100 |
| Betaine | Amino acid | 0.001 | 100 |
| L-cysteine | Amino acid | 0.001 | 100 |
| L-glutamic acid | Amino acid | 0.001 | 100 |
| L-glutamine | Amino acid | 0.001 | 100 |
| Glycine | Amino acid | 0.001 | 100 |
| L-histidine | Amino acid | 0.001 | 100 |
| Palmitoyl isoleucine | Amino acid | 0.001 | 100 |
| Leucine | Amino acid | 0.001 | 100 |
| L-lysine | Amino acid | 0.001 | 100 |
| Methionine | Amino acid | 0.001 | 100 |
| 3-Phenyl-L-alanine | Amino acid | 0.001 | 100 |
| Various plant based collagens | Amino acid | 0.001 | 100 |
| Proline | Amino acid | 0.001 | 100 |
| Pyrrolidone carboxylic acid | Amino acid | 0.001 | 100 |
| Serine | Amino acid | 0.001 | 100 |
| Sodium pyrrolidone | Amino acid | 0.001 | 100 |
| carboxylic acid | | | |
| Threonine | Amino acid | 0.001 | 100 |
| Tranexamic acid | Amino acid | 0.001 | 100 |
| Cetyl tranexamate mesylate | Amino acid | 0.001 | 100 |
| Tryptophan | Amino acid | 0.001 | 100 |
| L-tyrosine | Amino acid | 0.001 | 100 |
| Urea | Amino acid | 0.001 | 100 |
| Hydroxyethyl urea | Amino acid | 0.001 | 100 |
| Valine | Amino acid | 0.001 | 100 |

The above listed amino acids may act as a humectant or moisturizer in the cosmetic or pharmaceutical. It will be appreciated that the use of amino acids in may provide additional benefits. For instance, tranexamic acid and cetyl tranexamate mesylate may act as a brightening agent, and plant collagens are commonly used amino acid based proteins.

The at least one active ingredient may also be a probiotic. The table below is a nonexhaustive list of probiotics that may be used in the rapidly dissolving article.

| | Classifi- | Min. | Max. |
|---|---|---|---|
| Ingredient | cation | Range (%) | Range (%) |
| Bifida ferment lysate | Bacteria | 0.001 | 100 |
| Lactobacillus ferment lysate filtrate | Bacteria | 0.001 | 100 |
| Nitrosomonas eutropha | Bacteria | 0.001 | 100 |
| Saccharomyces boulardii | Bacteria | 0.001 | 100 |
| Vitreoscilla ferment | Bacteria | 0.001 | 100 |

The at least one active ingredient may also be a polysaccharide, monosaccharide, disaccharide, or oligosaccharide. The table below is a nonexhaustive list of saccharides that may be used in the rapidly dissolving article.

| | | Min. | Max. |
|---|---|---|---|
| Ingredient | Classification | Range (%) | Range (%) |
| Trehalose | Dissacharide | 0.001 | 100 |
| Fructose | Monosaccharide | 0.001 | 100 |
| Glucose | Monosaccharide | 0.001 | 100 |
| Fructooligosaccharides | Oligosaccharide | 0.001 | 100 |
| Galactooligosaccharides | Oligosaccharide | 0.001 | 100 |
| Raffinose | Oligosaccharide | 0.001 | 100 |
| Chitosan | Polysaccharide | 0.001 | 100 |
| Dextran | Polysaccharide | 0.001 | 100 |
| Glucomannan | Polysaccharide | 0.001 | 100 |
| Inulin | Polysaccharide | 0.001 | 100 |

The saccharides may provide a variety of benefits to the cosmetic or pharmaceutical. For example, the Fructose, Glucose, Fructooligosaccharides, Galactooligosaccharides, Raffinose, Glucomannan, and Inulin may act as prebiotics. Dextran and Trehalose may act as a humectant and with Trehalose providing additional antioxidant benefits. Chitosan is a derivative of chitin that may act as a healing and collagen stimulating agent. It will be appreciated that other saccharides may be used in the formulation of the cosmetic or pharmaceutical, and that the saccharides may provide other or additional benefits.

The at least one active ingredient may also be a peptide. The peptide may be one of a neurotransmitter inhibitor peptide, a signal peptide, a carrier peptide, an enzyme-inhibiting peptide, or an antimicrobial peptide. The table below is a nonexhaustive list of peptides that may be used in the rapidly dissolving article.

| | | Min. | Max. |
|---|---|---|---|
| Ingredient | Classification | Range (%) | Range (%) |
| Acetyl Hexapeptide-8 | Neurotransmitter | 0.001 | 100 |
| | inhibitor peptide | | |
| Acetyl Octapeptide-1/-3 | Neurotransmitter | 0.001 | 100 |
| (SNAP 8) | inhibitor peptide | | |
| Antimicrobial peptides | Antimicrobial | 0.001 | 100 |
| | peptide | | |
| GHK-cu | Carrier peptide | 0.001 | 100 |
| Enzyme-inhibiting | Enzyme-inhibiting | 0.001 | 100 |
| peptides | peptides | | |
| GEKG | Signal peptide | 0.001 | 100 |
| Copper Tripeptide-1 | Carrier peptide | 0.001 | 100 |
| GHK-Cu | | | |
| Hexapeptide-11 | Signal peptide | 0.001 | 100 |
| Hexapeptide-2 | Peptide | 0.001 | 100 |
| Lipospondin | Signal peptide | 0.001 | 100 |
| Manganese Tripeptide-1 | Carrier peptide | 0.001 | 100 |
| SNAP-25 | Neurotransmitter | 0.001 | 100 |
| | inhibitor peptide | | |
| Palmitoyl Hexapeptide-12 | Signal peptide | 0.001 | 100 |
| Palmitoyl Pentapetide-4 | Signal peptide | 0.001 | 100 |
| Palmitoyl Tetrapeptide-7 | Signal peptide | 0.001 | 100 |
| Palmitoyl Tripeptide-1 | Signal peptide | 0.001 | 100 |
| Palmitoyl Tripeptide-5 | Signal peptide | 0.001 | 100 |
| Pentapeptide-18 | Neurotransmitter | 0.001 | 100 |
| | inhibitor peptide | | |
| Pentapeptide-3 | Neurotransmitter | 0.001 | 100 |
| | inhibitor peptide | | |
| PKEK | Signal peptide | 0.001 | 100 |
| Polyglutamic Acid (PGA/ | Peptide- Polymer | 0.001 | 100 |
| y-PGA) | of Glutamic Acid | | |
| SA1-III | Signal peptide | 0.001 | 100 |
| Porin | Signal peptide | 0.001 | 100 |
| Tripeptide-10 Citrulline | Signal peptide | 0.001 | 100 |
| Tripeptide-3 | Neurotransmitter | 0.001 | 100 |
| | inhibitor peptide | | |

The peptides may provide a variety of benefits to the cosmetic or pharmaceutical. For example, the signal peptides may stimulate collagen, elastin, laminin, hyaluronic acid, elafin, epidermal growth factor (EGF), thrombospondin I (THBS1), decorin, melanocyte-stimulating hormones (MSH), granulocyte-macrophage colony-stimulating factor (GM-CSF) and fibronectin. Enzyme-inhibiting peptides may inhibit an enzyme. As an example, these peptides may inhibit enzymes like tyrosinase, which stimulates skin darkening and matrix metalloproteases (MMPs 1, 2 and 9) that can degrade tissue. It will be appreciated that other peptides may be used in the formulation of the cosmetic or pharmaceutical, and that the peptides may provide other or additional benefits.

The at least one active ingredient may also be a plant extract or plant extract derivatives. The table below is a nonexhaustive list of plant extracts and derivatives that may be used in the rapidly dissolving article.

| | | Min. | Max. |
|---|---|---|---|
| | | Range | Range |
| Ingredient | Classification | (%) | (%) |
| Aloe Barbadensis Leaf Juice | Aloe Vera | 0.001 | 100 |
| | Plant Extract | | |
| Aloe Barbadensis | Aloe Vera | 0.001 | 100 |
| | Plant Extract | | |
| Aloe Barbadensis Leaf | Aloe Vera | 0.001 | 100 |
| Extract | Plant Extract | | |
| Aloe Barbadensis Leaf | Aloe Vera | 0.001 | 100 |
| Juice Extract | Plant Extract | | |
| Aloe Barbadensis Leaf | Aloe Vera | 0.001 | 100 |
| Juice Powder | Plant Extract | | |
| Aloe Extract | Aloe Vera | 0.001 | 100 |
| | Plant Extract | | |
| Aloe Juice | Aloe Vera | 0.001 | 100 |
| | Plant Extract | | |
| Aloe Vera | Aloe Vera | 0.001 | 100 |
| | Plant Extract | | |
| Asparagus Officinalis Extract | Plant Extract | 0.001 | 100 |
| (Asparagus extract) | | | |
| Musa Spp Extract (Banana extract) | Plant Extract | 0.001 | 100 |
| Hordeum vulgare Extract (Barley | Plant Extract | 0.001 | 100 |
| extract) | | | |
| Centella Asiatica Extract | Plant Extract | 0.001 | 100 |
| Gotu Kola Extract | Plant Extract | 0.001 | 100 |
| Linum Usitatissimum Seed Extract | Plant Extract | 0.001 | 100 |
| (Flax seed extract) | | | |
| Allium Sativum Bulb Extract (Garlic | Plant Extract | 0.001 | 100 |
| extract) | | | |
| Camellia Sinensis Leaf | Plant Extract | 0.001 | 100 |
| Extract (Green tea extract) | | | |
| Camellia Sinensis Extract (Green | Plant Extract | 0.001 | 100 |
| tea extract) | | | |
| Licorice Extract | Plant Extract | 0.001 | 100 |
| Glycyrrhizic Acid (Licorice extract) | Plant Extract | 0.001 | 100 |
| Glycyrrhiza Glabra Root Extract | Plant Extract | 0.001 | 100 |
| (Licorice root extract) | | | |
| Glabridin (Licorice root extract) | Plant Extract | 0.001 | 100 |
| Avena Sativa Kernel Extract (Oat | Plant Extract | 0.001 | 100 |
| extract) | | | |
| Allium Cepa Bulb Extract (Onion | Plant Extract | 0.001 | 100 |
| extract) | | | |
| Opuntia ficus-indica extract (Prickly | Plant Extract | 0.001 | 100 |
| pear extract) | | | |
| Hydrolyzed Opuntia ficus-indica | Plant Extract | 0.001 | 100 |
| Flower extract (Prickly pear extract) | | | |
| Nephelium lappaceum Extract | Plant Extract | 0.001 | 100 |
| (Rambutan extract) | | | |
| Nephelium lappaceum Leaf Extract | Plant Extract | 0.001 | 100 |
| (Rambutan extract) | | | |
| Ganoderma Lucidum Extract (Reishi | Plant Extract | 0.001 | 100 |
| mushroom extract) | | | |
| Tremella Fuciformis Sporocarp | Plant Extract | 0.001 | 100 |
| Extract (Snow mushroom extract) | | | |
| Triticum Vulgare Bran Extract(Wheat | Plant Extract | 0.001 | 100 |
| bran extract) | | | |

The plant extracts may provide a variety of benefits to the cosmetic or pharmaceutical. For instance, the aloe vera derived extracts may be used as an emollient and humectant providing soothing, hydrating, and antioxidant benefits. The asparagus, banana, barley, flaxseed, garlic, oat, onion, and wheat bran extracts and their derivatives may act as prebiotics. The centella asiastic, gotu kola, green tea, and prickly pear extracts may act as antioxidants and provide soothing, hydrating, anti-inflammatory, and other antioxidant benefits. The licorice and licorice root extracts may also act as an antioxidant and provide anti-inflammatory and anti-microbial benefits. The rambutan extracts may act as plant based retinol alternatives. Finally, the reishi and snow mushroom extracts may provide anti-aging and hydrating benefits. It will be appreciated that other plant extracts or their derivatives may be used in the formulation of the cosmetic or pharmaceutical, and that the plant extracts and their derivatives may provide other or additional benefits.

The at least one active ingredient may also be a sphingolipid. Sphingolipids are a form of naturally occurring lipids that may be used as an emollient in the cosmetic or pharmaceutical thereby providing moisturizing benefits. The table below is a nonexhaustive list of sphingolipids that may be used in the rapidly dissolving article.

| Ingredient | Classification | Min. Range (%) | Max. Range (%) |
|---|---|---|---|
| Ceramide AP | Sphingolipids | 0.001 | 100 |
| Ceramide EOP | Sphingolipids | 0.001 | 100 |
| Ceramide EOS | Sphingolipids | 0.001 | 100 |
| Ceramide NG | Sphingolipids | 0.001 | 100 |
| Ceramide NP | Sphingolipids | 0.001 | 100 |
| Ceramide NS | Sphingolipids | 0.001 | 100 |

The at least one active ingredient may also be an ultraviolet (UV) blocker or filter. The UV blockers may be added to the cosmetic or pharmaceutical to protect the skin from harmful UV light, for instance in sunscreen products. The table below is a nonexhaustive list of UV blockers or filters that may be used in the rapidly dissolving article. It will be appreciated that other UV blockers or filters may be used in the formulation of the cosmetic or pharmaceutical, and that the UV blockers may provide additional benefits.

| | | Min. Range | Max. Range |
|---|---|---|---|
| Ingredient | Classification | (%) | (%) |
| Avobenzone | UV Blocker/Filter | 0.001 | 100 |
| Homosalate | UV Blocker/Filter | 0.001 | 100 |
| Mexoryl SX | UV Blocker/Filter | 0.001 | 100 |
| Octinoxate | UV Blocker/Filter | 0.001 | 100 |
| Octisalate | UV Blocker/Filter | 0.001 | 100 |
| Octocrylene | UV Blocker/Filter | 0.001 | 100 |
| Oxybenzone | UV Blocker/Filter | 0.001 | 100 |
| PABA | UV Blocker/Filter | 0.001 | 100 |
| Tinosorb M | UV Blocker/Filter | 0.001 | 100 |
| Tinosorb S | UV Blocker/Filter | 0.001 | 100 |
| Titanium dioxide | UV Blocker/Filter | 0.001 | 100 |
| Trolamine | UV Blocker/Filter | 0.001 | 100 |
| Salicylate PABA | | | |
| Zinc oxide | UV Blocker/Filter | 0.001 | 100 |

The at least one active ingredient may also be a vitamin or nutrient or vitamin or nutrient derivative. The table below is a nonexhaustive list of vitamins, nutrients, and derivatives filters that may be used in the rapidly dissolving article.

| | | Min. | Max. |
|---|---|---|---|
| | Classifi- | Range | Range |
| Ingredient | cation | (%) | (%) |
| Ascorbic acid (Vitamin C) | Vitamin | 0.001 | 100 |
| Ascorbyl Glucoside (Vitamin C | Vitamin | 0.001 | 100 |
| derivative) | | | |
| Ethylated L-Ascorbic Acid | Vitamin | 0.001 | 100 |
| (Vitamin C derivative) | | | |
| L-Ascorbic acid (Vitamin C | Vitamin | 0.001 | 100 |
| derivative) | | | |
| Magnesium Ascorbyl Phosphate | Vitamin | 0.001 | 100 |
| (Vitamin C derivative) | | | |
| Niacinamide | Vitamin | 0.001 | 100 |
| B-panthenol (Form of Vitamin | Vitamin | 0.001 | 100 |
| B5) | | | |
| Sodium Ascorbyl Phosphate | Vitamin | 0.001 | 100 |
| (Vitamin C derivative) | | | |
| Tetrahexyldecyl Ascorbate | Vitamin | 0.001 | 100 |
| (Vitamin C derivative) | | | |
| DL-alpha-tocopheryl acetate | Vitamin | 0.001 | 100 |
| (Vitamin E derivative) | | | |
| D-alpha-tocopherol (Vitamin E | Vitamin | 0.001 | 100 |
| derivative) | | | |
| DL-Alpha Tocopheryl Acetate | Vitamin | 0.001 | 100 |
| (Vitamin E derivative) | | | |
| Tocopheryl glucoside (Vitamin E | Vitamin | 0.001 | 100 |
| derivative) | | | |
| Tocopheryl phosphate (Vitamin E | Vitamin | 0.001 | 100 |
| derivative) | | | |
| Tocopheryl acetate (Vitamin E | Vitamin | 0.001 | 100 |
| derivative) | | | |
| Tocopherol acetate (Vitamin E | Vitamin | 0.001 | 100 |
| derivative) | | | |
| Vitamin E acetate (Vitamin E | Vitamin | 0.001 | 100 |
| derivative) | | | |
| Hydroxypinacolone Retinoate | Vitamin - | 0.001 | 100 |
| (An ester of retinoic acid) | Retinoid | | |
| Retinol (Vitamin A molecule) | Vitamin - | 0.001 | 100 |
| | Retinoid | | |
| Retinaldehyde (A stabilized form | Vitamin - | 0.001 | 100 |
| of vitamin A) | Retinoid | | |
| Adapalene (a topical retinoid) | Vitamin - | 0.001 | 100 |
| | Retinoid | | |
| Retinoic Acid (active biological | Vitamin - | 0.001 | 100 |
| form of vitamin A) | Retinoid | | |
| Retinal (retinol derivative) | Vitamin - | 0.001 | 100 |
| | Retinoid | | |
| Tazarotene (a topical retinoid) | Vitamin - | 0.001 | 100 |
| | Retinoid | | |
| Retinyl Palmitate (combination of | Vitamin - | 0.001 | 100 |
| retinol and palmitic acid) | Retinol Ester | | |
| Retinyl Linoleate (retinol | Vitamin - | 0.001 | 100 |
| derivative) | Retinol Ester | | |
| Retinyl Propioniate (retinol ester | Vitamin - | 0.001 | 100 |
| with retinol and propionic acid | Retinol Ester | | |
| combined) | | | |

The above nutrients and vitamins may provide a variety of benefits as part of the formulation of the cosmetic or pharmaceutical. For instance, B-panthenol may act as a humectant and moisturizer and the vitamin E derivatives may act as skin conditioners. It will be appreciated that other vitamins and nutrients and their derivatives may be used in the formulation of the cosmetic or pharmaceutical, and that the vitamins, nutrients, and derivatives may provide additional cosmetic or pharmaceutical benefits.

The at least one active ingredient may also be an antioxidant. The table below is a nonexhaustive list of antioxidants that may be used in the rapidly dissolving article.

| | | Min. Range | Max. Range |
|---|---|---|---|
| Ingredient | Classification | (%) | (%) |
| Astaxanthin | Carotenoid - | 0.001 | 100 |
| | Antioxidant | | |
| Alpha Arbutin | Glycoside - | 0.001 | 100 |
| | Antioxidant | | |
| Beta Arbutin | Glycoside - | 0.001 | 100 |
| | Antioxidant | | |
| 4-butylresorcinol | Phenol - | 0.001 | 100 |
| | Antioxidant | | |
| Resveratrol | Phenol - | 0.001 | 100 |
| | Antioxidant | | |
| Ferulic Acid | Phenolic acid - | 0.001 | 100 |
| | Antioxidant | | |
| Diglucosyl Gallic Acid | Phenolic acid - | 0.001 | 100 |
| | Antioxidant | | |
| Bakuchiol | Terpenophenol - | 0.001 | 100 |
| | Antioxidant | | |

The use of these antioxidants in the formulation of the cosmetic or pharmaceutical may provide skin protection and skin-brightening benefits in addition to other antioxidant benefits. For instance, 4-butylresorcinol and resveratrol may act as a tyrosinas inhibitor and diglucosyl gallic acid may act as a skin-brightening agent. It will be appreciated that other antioxidants and their derivatives may be used in the formulation of the cosmetic or pharmaceutical, and that the antioxidants may provide additional cosmetic or pharmaceutical benefits.

The at least one active ingredient may also be an enzyme or coenzyme. The table below is a nonexhaustive list of enzyme or coenzyme that may be used in the rapidly dissolving article.

| | | Min. Range | Max. Range |
|---|---|---|---|
| Ingredient | Classification | (%) | (%) |
| Ubiquinone (Coenzyme | Coenzyme | 0.001 | 100 |
| Q10 (CoQ10)) | | | |
| Ubiquinol (Coenzyme Q10 | Coenzyme | 0.001 | 100 |
| (CoQ10)) | | | |
| Lactococcus Ferment | Enzyme | 0.001 | 100 |
| Lysate (Lysate) | | | |

The use of these enzymes and coenzymes in the formulation of the cosmetic or pharmaceutical may provide benefits. First, instance lysate may act as a postbiotic, and coenzyme Q10 may act as an antioxidant that protects that skin. It will be appreciated that other enzymes and coenzymes may be used in the formulation of the cosmetic or pharmaceutical, and that the enzymes and coenzymes may provide additional cosmetic or pharmaceutical benefits.

The at least one active ingredient may also be a protein. The table below is a nonexhaustive list of proteins that may be used in the rapidly dissolving article. The table includes amino acid based proteins that provide cosmetic or pharmaceutical benefits. It will be appreciated that other proteins may be used in the formulation of the cosmetic or pharmaceutical.

| | | Min. Range | Max. Range |
|---|---|---|---|
| Ingredient | Classification | (%) | (%) |
| Tripeptide-29 (AC Collagen | Protein | 0.001 | 100 |
| Prepeptide | | | |
| Hydrolyzed Collagen | Protein | 0.001 | 100 |
| Collagen Peptides | Protein | 0.001 | 100 |

The at least one active ingredient may also be a polyol. The table below is a nonexhaustive list of polyols that may be used in the rapidly dissolving article. The polyols may act as a humectant or moisturizer in the cosmetic or pharmaceutical. It will be appreciated that other polyols may be used in the formulation of the cosmetic or pharmaceutical, and that the polyols may provide additional cosmetic or pharmaceutical benefits.

| Ingredient | Classification | Min. Range (%) | Max. Range (%) |
|---|---|---|---|
| Glycerin | Polyol | 0.001 | 100 |
| Mannitol | Polyol | 0.001 | 100 |

The at least one active ingredient may also be a form of hyaluronic acid. Hyaluronic acid is a polymer that may act as a humectant and provide moisturizing benefits in the cosmetic or pharmaceutical. The table below is a nonexhaustive list of the forms of hyaluronic acid that may be used in the rapidly dissolving article.

| | Classifi- | Min. Range | Max. Range |
|---|---|---|---|
| Ingredient | cation | (%) | (%) |
| Hyaluronic acid | Polymer | 0.001 | 100 |
| Hydrolyzed Sodium Hyaluronate | Polymer | 0.001 | 100 |
| (hydrolyzed hyaluronic acid salt) | | | |
| Sodium Hyaluronate (salt form | Polymer | 0.001 | 100 |
| of hyaluronic acid) | | | |
| Hydrolyzed hyaluronic acid | Polymer | 0.001 | 100 |
| Sodium Hyaluronate | Polymer | 0.001 | 100 |
| Crosspolymer (hyaluronic acid | | | |
| attached to a polymer structure) | | | |
| Sodium Acetylated Hyaluronate | Polymer | 0.001 | 100 |
| (acetyl ester of sodium | | | |
| hyaluronate) | | | |

The at least one active ingredient may also be a various chemical or organic compound. The table below is a nonexhaustive list of these compounds that may be used in the rapidly dissolving article.

| | | Min. | Max. |
|---|---|---|---|
| | | Range | Range |
| Ingredient | Classification | (%) | (%) |
| Allantoin | Chemical | 0.001 | 100 |
| | compound | | |
| Alcloxa (Aluminium | Chemical | 0.001 | 100 |
| chlorhydroxy allantoinate) (an | compound | | |
| allantoin derivative) | | | |
| Aldioxa (Aluminium | Chemical | 0.001 | 100 |
| dihydroxy allantoinate) (an | compound | | |
| allantoin derivative) | | | |
| Potassium Azeloyl Diglycinate | Chemical | 0.001 | 100 |
| (Azeloyl Acid) | compound | | |
| Benzoyl peroxide | Chemical | 0.001 | 100 |
| | compound | | |
| Azelaic Acid | Dicarboxylic | 0.001 | 100 |
| | acids | | |
| 1;3 propanediol (Propanediol) | Glycol | 0.001 | 100 |
| Coco-caprylate/Caprate | Mixed Ester | 0.001 | 100 |
| 4-hexylresorcinol | Organic Compound | 0.001 | 100 |
| (Hexylresorcinol) | | | |
| Hydroquinone | Organic Compound | 0.001 | 100 |
| Kojic Acid | Organic Compound | 0.001 | 100 |
| Sodium Lactate | Organic Compound | 0.001 | 100 |
| Xylitol | Polyalcohol | 0.001 | 100 |

The use of these various compounds in the formulation of the cosmetic or pharmaceutical may provide a variety of benefits. For instance, allantoin and its derivatives are a plant extract that may provide soothing benefits. Azeloyl acid, benzoyl peroxide, and azelaic acid may provide anti-inflammatory and anti-microbial benefits. Propanediol may has a humectant in the cosmetic or pharmaceutical. Hexylresorcinol, Hydroquinone, and Kojic Acid may as a skin brightening agent and antioxidant. Finally, xylitol may act as a probiotic in the cosmetic or pharmaceutical.

The rapidly dissolving article may also include excipients, or inactive ingredients, in addition to the active ingredients. Exemplary excipients will be discussed in the following paragraphs. Before being dissolved into the liquid preparation, the excipients or their derivatives may be in a pure, blended, liposomal, or hydrolyzed form. A nonexhaustive list of the types of excipients that may be used in lyophilized cosmetics or pharmaceuticals includes: amino acids, sugars, saccharides, polyols, polymers, surfactants, buffers, chelating complexes, and inorganic salts. It will be appreciated that one skilled in the art may recognize that other types of excipients may be used in the liquid preparation. The below listed minimum and maximum ranges may be the percentage amount of the excipients in the liquid preparation if that excipient is used in the formulation of the cosmetic or pharmaceutical. It will be appreciated while the below tables group ingredients together, many of these ingredients may fit into multiple of the below types of active ingredients.

The excipient may be an amino acid. The table below is a nonexhaustive list of amino acids that may be used in the rapidly dissolving article as an excipient.

| Ingredient | Classification | Min. Range (%) | Max. Range (%) |
|---|---|---|---|
| Arginine | Amino Acid | 0.001 | 50 |
| Glycine | Amino Acid | 0.001 | 50 |
| Histidine | Amino Acid | 0.001 | 50 |
| Various Plant | Amino Acid | 0.001 | 50 |
| Collagens | | | |
| Potassium acetate | Amino Acid | 0.001 | 50 |

The use of these amino acids may provide various functions in the formulation of the cosmetic or pharmaceutical. For instance, arginine, glycine, histidine, and plant collagens may act as a bulking agent. Arginine and potassium acetate may act as a buffering agent. It will be appreciated that other amino acids may be used in the formulation of the cosmetic or pharmaceutical, and that the amino acids may provide additional or alternative functions.

The excipient may be an alcohol. The table below is a nonexhaustive list of alcohols that may be used in the rapidly dissolving article as an excipient.

| | | Min. Range | Max. Range |
|---|---|---|---|
| Ingredient | Classification | (%) | (%) |
| Benxyl Alcohol (an | Alcohol | 0.001 | 50 |
| Alpha hydroxy acid | | | |
| extracted from milk) | | | |
| Ethanol | Alcohol | 0.001 | 50 |
| Isopropyl alcohol | Alcohol | 0.001 | 50 |
| Tert-butyl alcohol | Alcohol | 0.001 | 50 |

The alcohols may act as a solubilizing agent or co-solvent in the formulation of the cosmetic or pharmaceutical. It will be appreciated that other alcohols may be used in the formulation of the cosmetic or pharmaceutical, and that the alcohols may provide additional or alternative functions.

The excipient may also be a polysaccharide, monosaccharide, disaccharide, or oligosaccharide. The table below is a nonexhaustive list of saccharides that may be used in the rapidly dissolving article.

| | | Min. Range | Max. Range |
|---|---|---|---|
| Ingredient | Classification | (%) | (%) |
| Lactose | Disaccharide | 0.001 | 50 |
| Sucrose | Disaccharide | 0.001 | 50 |
| Sucrose | Disaccharide | 0.001 | 50 |
| Dextrose | Monosaccharide | 0.001 | 50 |
| Glucose | Monosaccharide | 0.001 | 50 |
| Alpha cyclodextrin | Oligosaccharides | 0.001 | 50 |
| Hydroxypropyl-β- | Oligosaccharides | 0.001 | 50 |
| cyclodextrin | | | |
| Algin - Alginic Acid/ | Polysaccharide | 0.001 | 50 |
| Alginate/Sodium | | | |
| Alginate | | | |
| Chitosan | Polysaccharide | 0.001 | 50 |
| Dextran | Polysaccharide | 0.001 | 50 |
| Ficoll (derivative of | Polysaccharide | 0.001 | 50 |
| chitan) | | | |
| Tara Gum | Polysaccharide | 0.001 | 50 |
| (Caesalpinia spinosa | | | |
| gum) | | | |
| Raffinose | Trisaccharide | 0.001 | 50 |

The use of these saccharides may provide various functions in the formulation of the cosmetic or pharmaceutical. Lactose, sucrose, glucose, dextran, and raffinose may act as a bulking agent. Sucrose and glucose may also act as a tonicifying agent. Alpha cyclodextrin and hydroxypropyl-ß-cyclodextrin may act as a solubilizing agent and a complexing agent. Algin and tara gum may act as a thickening agent. Dextran may also act as a binding agent. Chitosan may act as a delivery agent. Tara gum may also act as an emulsifier, a stabilizer, a texturing agent, or a collapse or temperature modifier. It will be appreciated that other saccharides may be used in the formulation of the cosmetic or pharmaceutical, and that the saccharides may provide additional or alternative functions.

The excipient may also be a polyol. The table below is a nonexhaustive list of polyols that may be used in the rapidly dissolving article.

| Ingredient | Classification | Min. Range (%) | Max. Range (%) |
|---|---|---|---|
| Glycerol/ | Polyol | 0.001 | 50 |
| Glycerin | | | |
| Mannitol | Polyol | 0.001 | 50 |
| Meglumine | Polyol | 0.001 | 50 |
| Sorbitol | Polyol | 0.001 | 50 |
| Polyglyceryl-4 | Polyglycerol | 0.001 | 50 |
| Caprate | | | |

Polyols may provide various functions in the formulation of the cosmetic or pharmaceutical. The glycerol/glycerin, meglumine, and polyglyceryl-4 caprate (a fatty acid found in coconut milk) may act as a solubilizing agent. Mannitol may act as a lyoprotectant, binding agent, or ph adjuster. It will be appreciated that other polyols may be used in the formulation of the cosmetic or pharmaceutical, and that the polyols may provide additional or alternative functions.

The excipient may also be a glycol. The table below is a nonexhaustive list of glycols that may be used in the rapidly dissolving article.

| | | Min. Range | Max. Range |
|---|---|---|---|
| Ingredient | Classification | (%) | (%) |
| 1;3 propanediol | Glycol | 0.001 | 50 |
| (Propanediol) | | | |
| 1,2-Propylene glycol | Glycol | 0.001 | 50 |
| (Propylene glycol) | | | |

Glycols may provide various functions in the formulation of the cosmetic or pharmaceutical. For instance, propanediol may act as a preservative, a solvent or an emollient. Propylene glycol may act as a solvent. It will be appreciated that other polyols may be used in the formulation of the cosmetic or pharmaceutical, and that the polyols may provide additional or alternative functions.

The excipient may also be a polymer. The table below is a nonexhaustive list of polymers that may be used in the rapidly dissolving article.

| | | Min. Range | Max. Range |
|---|---|---|---|
| Ingredient | Classification | (%) | (%) |
| Sodium carboxymethyl | Polymer | 0.001 | 50 |
| cellulose (Cellulose gum) | | | |
| Sodium Hyaluronate | Polymer | 0.001 | 50 |
| (Hyaluronic acid) | | | |
| Polyethylene glycol | Polymer | 0.001 | 50 |

Polymers may provide various functions in the formulation of the cosmetic or pharmaceutical. For instance, the polyethylene glycol, hyaluronic acid, and dextran may be used as a bulking agent. Cellulose gum, a natural polymer, may act as a texturizing agent. Polyethylene glycol, a water-soluble carrier substance, may also act as an emulsifier or an emollient. It will be appreciated that other polymers may be used in the formulation of the cosmetic or pharmaceutical, and that the polymers may provide additional or alternative functions.

The excipient may also be an organic compound or acid. The table below is a nonexhaustive list of polymers that may be used in the rapidly dissolving article.

| | | Min. | Max. |
|---|---|---|---|
| | | Range | Range |
| Ingredient | Classification | (%) | (%) |
| Citric Acid | Organic Acid | 0.001 | 50 |
| Potassium citrate (potassium | Organic Acid | 0.001 | 50 |
| salt of citric acid) | | | |
| Sodium citrate (sodium salt of | Organic Acid | 0.001 | 50 |
| citric acid) | | | |
| Tartaric acid | Organic Acid | 0.001 | 50 |
| 2-propanone or dimethyl ketone | Organic Compound | 0.001 | 50 |
| (acetone) | | | |
| Phenol | Organic Compound | 0.001 | 50 |
| Sodium acetate | Organic Compound | 0.001 | 50 |
| Sodium Lactate | Organic Compound | 0.001 | 50 |
| Tris acetate | Organic Compound | 0.001 | 50 |
| Tris(hydroxymethyl)amino- | Organic Compound | 0.001 | 50 |
| methane (Tris base) | | | |
| Tris Hydrochloride | Organic Compound | 0.001 | 50 |
| Methylene chloride | Organochlorine | 0.001 | 50 |
| M-cresol | Phenol | 0.001 | 50 |

Organic compounds and acids may provide various functions in the formulation of the cosmetic or pharmaceutical. For instance, citric acid, potassium acid, sodium acid, tartaric acid, sodium acetate, sodium lactate, tris acetate, tris base, and tris hydrochloride may act as a buffering agent. Tris hydrochloride and M-cresol may act as an anti-microbial agent. Methylene chloride and acetone may act as a solubilizing agent. It will be appreciated that other organic compounds may be used in the formulation of the cosmetic or pharmaceutical, and that the organic compounds may provide additional or alternative functions.

The excipient may also be a protein. As an example, the cosmetic or pharmaceutical formulation may include collagen or gelatin in the range of 0.001-50.0 percent of the liquid preparation. The gelatin may act as a collapse or temperature modifier. The collagen may act as a bulking agent. It will be appreciated that other proteins may be used in the formulation of the cosmetic or pharmaceutical, and that the proteins may provide additional or alternative functions.

The excipient may also be an alpha hydroxy acid. As an example, the cosmetic or pharmaceutical may include lactic acid in the range of 0.001-50.0 percent of the liquid preparation. The lactic acid may act as a ph balancer or an anti-microbial agent. It will be appreciated that other AHAs may be used in the formulation of the cosmetic or pharmaceutical, and that the AHAs may provide additional or alternative functions.

The excipient may also be a hydroxybenzoic acid. As an example, the cosmetic or pharmaceutical may include ethyl paraben or methyl paraben in the range of 0.001-50.0 percent of the liquid preparation acting as an anti-microbial agent. It will be appreciated that other hydroxybenzoic acids may be used in the formulation of the cosmetic or pharmaceutical, and that the hydroxybenzoic acids may provide additional or alternative functions.

The excipient may also be a mixed ester. As an example, the cosmetic or pharmaceutical may include Caprylic/Capric Triglyceride, a plant extract or natural fatty acid, or Coco-caprylate/Caprate, an ester of natural fatty alcohol, caprylic acid and capric acid, in the range of 0.001-50.0 percent of the liquid preparation acting as an anti-microbial agent. Caprylic/capric triglyceride may act as an emollient. Coco-caprylate/Caprate may act as a solubilizing or conditioning agent. It will be appreciated that other mixed esters may be used in the formulation of the cosmetic or pharmaceutical, and that the mixed esters may provide additional or alternative functions.

The pharmaceutical or cosmetic may include other various chemical compounds or solvents as an excipient. The table below is a nonexhaustive list of other various chemical compounds or solvents that may be used in the rapidly dissolving article.

| | | Min. Range | Max. Range |
|---|---|---|---|
| Ingredient | Classification | (%) | (%) |
| Polysorbate 20 | Chemical compound | 0.001 | 50 |
| Polysorbate 80 | Chemical compound | 0.001 | 50 |
| Sodium chloride | Chemical compound | 0.001 | 50 |
| Sodium phosphate | Chemical compound | 0.001 | 50 |
| Sodium hydroxide | Inorganic compound | 0.001 | 50 |
| Zinc chloride | Ionic salt | 0.001 | 50 |
| Hydrochloric acid | Mineral acid | 0.001 | 50 |
| Ethoxydiglycol | Solvent | 0.001 | 50 |

Chemical compounds or solvents may provide various functions in the formulation of the cosmetic or pharmaceutical. For example, polysorbate 20 and polysorbate 80 may act as an emulsifier or a surfactant. Sodium phosphate and sodium hydroxide may be a buffering agent. Sodium phosphate may be a tonicifying agent. Sodium hydroxide, hydrochloric acid (a ph adjuster), and ethoxydiglycol may be solubilizing agents. It will be appreciated that other chemical compounds may be used in the formulation of the cosmetic or pharmaceutical, and that the chemical compounds may provide additional or alternative functions.

The cosmetic or pharmaceutical's formula for the liquid preparation may include any combination of the previously listed at least one active ingredient and excipients. Below are exemplary formulas of active ingredients and excipients that may be used in the liquid preparation. Other formulas may be used in the liquid preparation to create the rapidly dissolving article.

A first exemplary formula may include Alpha Arbutin, and either Alginate, Chitosan, Sodium Hyaluronate (Hyaluronic Acid), or Collagen as a base. One embodiment of the first exemplary formula may include the ingredients in the following first table at the listed weight percentages before the liquid preparation is freeze-dried and in the second table at the listed weight percentages after the liquid preparation is freeze-dried.

First Exemplary Formula Before Freeze-Drying

| | | | % in liquid |
|---|---|---|---|
| Trade name | Ingredients | % WT | preparation |
| Distilled Water | Distilled | 96.89% | 96.89% |
| | Water | | |
| Alpha Arbutin 99% | Alpha | 2.00% | 2.11% |
| | Arbutin | | |
| | Water | 0.11% | |
| Algogel VCG 1321 | Algin | 0.90% | 1.00% |
| | Water | 0.10% | |

First Exemplary Formula After Freeze-Drying

| Trade name | Ingredients | % WT |
|---|---|---|
| Water | Distilled | 10.0% |
| | Water | |
| Alpha Arbutin 99% | Alpha | 62.11% |
| | Arbutin | |
| Algogel VCG 1321 | Algin | 27.89% |

A second exemplary formula may include potassium Azeloyl Diglycinate (azelaic acid), and either Alginate, Chitosan, Sodium Hyaluronate (Hyaluronic Acid), or Collagen as a base. One embodiment of the second exemplary formula may include the ingredients in the following first table at the listed weight percentages before the liquid preparation is freeze-dried and in the second table at the listed weight percentages after the liquid preparation is freeze-dried.

Second Exemplary Formula Before Freeze-Drying

| | | | % in liquid |
|---|---|---|---|
| Trade name | Ingredients | % WT | preparation |
| Distilled Water | Distilled Water | 93.11% | 93.11% |
| Azeclair P | Potassium Azeloyl | 5.00% | 5.39% |
| | Diglycinate | | |
| | Water | 0.39% | |
| Algogel VCG 1321 | Algin | 1.35% | 1.50% |
| | Water | 0.15% | |

Second Exemplary Formula After Freeze-Drying

| Trade name | Ingredients | % WT |
|---|---|---|
| Water | Distilled Water | 10.00% |
| Azeclair P | Potassium Azeloyl | 70.90% |
| | Diglycinate | |
| Algogel VCG 1321 | Algin | 19.10% |

A third exemplary formula may include Niacinamide USP, and either Alginate, Chitosan, Sodium Hyaluronate (Hyaluronic Acid), or Collagen as a base. One embodiment of the third exemplary formula may include the ingredients in the following first table at the listed weight percentages before the liquid preparation is freeze-dried and in the second table at the listed weight percentages after the liquid preparation is freeze-dried.

Third Exemplary Formula Before Freeze-Drying

| | | | % in liquid |
|---|---|---|---|
| Trade name | Ingredients | % WT | preparation |
| Distilled water | Distilled | 97.0% | 97.0% |
| | Water | | |
| Macamin NC/ | Niacinamide | 2.0% | 2.002% |
| Niacinamide USP | Water | 0.002% | |
| Algogel VCG 1321 | Algin | 0.90% | 1.0% |
| | Water | 0.10% | |

Third Exemplary Formula After Freeze-Drying

| Trade name | Ingredients | % WT |
|---|---|---|
| Distilled Water | Distilled | 10.0% |
| | Water | |
| Niacinamide | Niacinamide | 62.11% |
| | USP | |
| Algogel VCG 1321 | Algin | 27.89% |

A fourth exemplary formula may include Gluconolactone USP, Dextran, Maltobionic Acid, Lactobionic Acid, Aloe Barbadensis (Aloe Vera) Leaf Juice, and either Alginate, Chitosan, Sodium Hyaluronate (Hyaluronic Acid), or Collagen as a base. One embodiment of the fourth exemplary formula may include the ingredients in the following first table at the listed weight percentages before the liquid preparation is freeze-dried and in the second table at the listed weight percentages after the liquid preparation is freeze-dried.

Fourth Exemplary Formula Before Freeze-Drying

| | | | % in liquid |
|---|---|---|---|
| Trade name | Ingredients | % WT | preparation |
| Distilled Water | Distilled Water | 79.409% | 79.409% |
| SMA'RT | Gluconolactone | 2.000% | 2.004% |
| Gluconolactone USP | Water | 0.004% | |
| Maltobionic acid | Maltobionic acid | 1.000% | 1.040% |
| | Water | 0.040% | |
| Lactobionic Acid | Lactobionic Acid | 1.000% | 1.037% |
| | Water | 0.037% | |
| Aloe Freeze Dried Powder | Aloe Barbadensis | 0.010% | 0.0103% |
| (100:1) | Leaf Juice | | |
| | Water | 0.0003% | |
| Algogel VCG 1321 | Algin | 0.898% | 1.000% |
| | Water | 0.102% | |
| Dextran from Leuconostoc | Dextran | 2.910% | 3.000% |
| spp. Mr ~40,000 | Water | 0.090% | |
| Sodium Hydroxide | Sodium | 0.500% | 12.500% |
| Solution, 1M | hydroxide | | |
| | Water | 12.000% | |

Fourth Exemplary Formula After Freeze-Drying

| Ingredients | % WT | % WT |
|---|---|---|
| Distilled Water | Distilled Water | 10.0% |
| SMA'RT Gluconolactone USP | Gluconolactone | 21.640% |
| Maltobionic acid | Maltobionic acid | 10.820% |
| Lactobionic Acid | Lactobionic Acid | 10.820% |
| Aloe Freeze Dried Powder (100:1) | Aloe Barbadensis | 0.108% |
| | Leaf Juice | |
| Algogel VCG 1321 | Algin | 9.716% |
| Dextran from Leuconostoc spp. | Dextran | 31.486% |
| Mr ~40,000 | | |
| Sodium Hydroxide Solution, 1M, | Sodium hydroxide | 5.410% |

A fifth exemplary formula may include Sodium Hyaluronate (Hyaluronic Acid), Betaine, Polyglutamic Acid, and either Alginate, Chitosan, Sodium Hyaluronate (Hyaluronic Acid), or Collagen as a base.

A sixth exemplary formula may include Centella asiatica (Cica) Extract, Camellia Sinensis (Green Tea) Extract, and either Alginate, Chitosan, Sodium Hyaluronate (Hyaluronic Acid), or Collagen as a base.

A seventh exemplary formula may include Tripeptide-1 (and) Copper (Copper Peptide 1), Acetyl Hexapeptide-8, Palmitoyl Tripeptide-1, Palmitoyl Tetrapeptide-7, and either Alginate, Chitosan, Sodium Hyaluronate (Hyaluronic Acid), or Collagen as a base. One embodiment of the seventh exemplary formula may include the ingredients in the following first table at the listed weight percentages before the liquid preparation is freeze-dried and in the second table at the listed weight percentages after the liquid preparation is freeze-dried.

Seventh Exemplary Formula Before Freeze-Drying

| | | | % in liquid |
|---|---|---|---|
| Trade name | Ingredients | % WT | preparation |
| Distilled Water | Distilled Water | 89.37% | 89.37% |
| GHK-Cu | GHK-Cu | 0.5000% | 0.5388% |
| | water | 0.0388% | |
| Acetyl | Acetyl Hexapeptide-8 | 0.0101% | 0.0104% |
| Hexapeptide-8 | water | 0.0004% | |
| Palmitoyl | Palmitoyl Tripeptide-1 | 0.0500% | 0.0505% |
| Tripeptide-1 | water | 0.0005% | |
| Ritalac LA | Lactic acid | 1.2114% | 1.3750% |
| | water | 0.1636% | |
| Jeechem PDO | Propanediol | 3.3990% | 3.4% |
| | water | 0.0010% | |
| Palmitoyl | Palmitoyl Tetrapeptide-7 | 0.0500% | 0.051% |
| Tetrapeptide-7 | water | 0.0011% | |
| Dextran | Dextran Leuconostoc | 3.9% | 4.0% |
| | spp | | |
| | water | 0.1% | |
| Solagum TARA | Caesalpinia spinosa gum | 0.1850% | 0.2% |
| | water | 0.0150% | |
| PEG 4000 | Polyethylene glycol | 1.00% | 1.00% |
| | 4000 | | |

Seventh Exemplary Formula After Freeze-Drying

| Trade Name | Ingredient | % WT |
|---|---|---|
| Water | Distilled Water | 10.00% |
| GHK-Cu | GHK-Cu | 6.53% |
| Acetyl Hexapeptide-8 | Acetyl Hexapeptide-8 | 0.13% |
| Palmitoyl Tripeptide-1 | Palmitoyl Tripeptide-1 | 0.65% |
| Ritalac LA | Lactic acid | 15.83% |
| Jeechem PDO | Propanediol | 0.00% |
| Palmitoyl | Palmitoyl Tetrapeptide-7 | 0.65% |
| Tetrapeptide-7 | | |
| Dextran | Dextran | 50.71% |
| Solagum TARA | Caesalpinia spinosa gum | 2.42% |
| PEG 4000 | Polyethylene glycol | 13.07% |
| | 4000 | |

An eighth exemplary formula may include Glycolic Acid (Hydroxyacetic acid 99%), Lactic Acid, Phytic Acid, Mandelic Acid (DL-Mandelic Acid 99%), Salicylic Acid, and either Alginate, Chitosan, Sodium Hyaluronate (Hyaluronic Acid), or Collagen as a base.

A ninth exemplary formula may include L-Ascorbic acid, DL-Alpha Tocopheryl Acetate, Ferulic Acid, Propanediol or Ethoxydiglycol, and either Alginate, Chitosan, Sodium Hyaluronate (Hyaluronic Acid), or Collagen as a base.

A tenth exemplary formula may include Hydroxypinacolone Retinoate, DL-Alpha Tocopheryl Acetate, a solvent, and either Alginate, Chitosan, Sodium Hyaluronate (Hyaluronic Acid), or Collagen as a base. One embodiment of the tenth exemplary formula may include the ingredients in the following first table at the listed weight percentages before the liquid preparation is freeze-dried and in the second table at the listed weight percentages after the liquid preparation is freeze-dried.

Tenth Exemplary Formula Before Freeze-Drying

| | | | % in liquid |
|---|---|---|---|
| Trade name | Ingredients | % WT | preparation |
| Distilled Water | Distilled Water | 88.70% | 88.70% |
| Waglinol 20080 MB | Coco- | 5.0000% | 5.0000% |
| | caprylate/Caprate | | |
| Soldoc PG 410 MB | Polyglyceryl-4 | 1.8072% | 2.0000% |
| | Caprate | | |
| | water | 0.1928% | |
| Hydroxypinacolone | Hydroxypinacolone | 0.3000% | 0.3007% |
| Retinoate | Retinoate | | |
| | water | 0.0007% | |
| Vitamin E acetate | DL-alpha-tocopheryl | 1.0000% | 1.0000% |
| 98% Oil | acetate | | |
| Dextran | Dextran Leuconostoc | 1.9400% | 2.0% |
| | spp | | |
| | water | 0.0600% | |
| Algogel ® VCG 1321 | algin | 0.8980% | 1.000% |
| | water | 0.1020% | |

Tenth Exemplary Formula After Freeze-Drying

| Trade Name | Ingredients | % WT |
|---|---|---|
| Waglinol 20080 MB | Coco- | 36.55% |
| | caprylate/Caprate | |
| Distilled water | Distilled Water | 20.00% |
| Dextran | Dextran Leuconostoc | 14.18% |
| | spp | |
| Soldoc PG 410 MB | Polyglyceryl-4 Caprate | 13.21% |
| Vitamin E acetate 98% | DL-alpha-tocopheryl | 7.31% |
| Oil | acetate | |
| Algogel ® VCG 1321 | algin | 6.56% |
| Hydroxypinacolone | Hydroxypinacolone | 2.19% |
| Retinoate | Retinoate | |

Another aspect of this disclosure comprises a method of making the cosmetic or pharmaceutical including the above-mentioned active ingredients and excipients as a rapidly dissolving article, especially in the above noted formulations. For example, the rapidly dissolving article may be a tablet that can be rehydrated in water to obtain a single dose or use of the cosmetic or pharmaceutical. It will be appreciated that the formulas may be prepared by following current good manufacturing practice (CGMP) regulations.

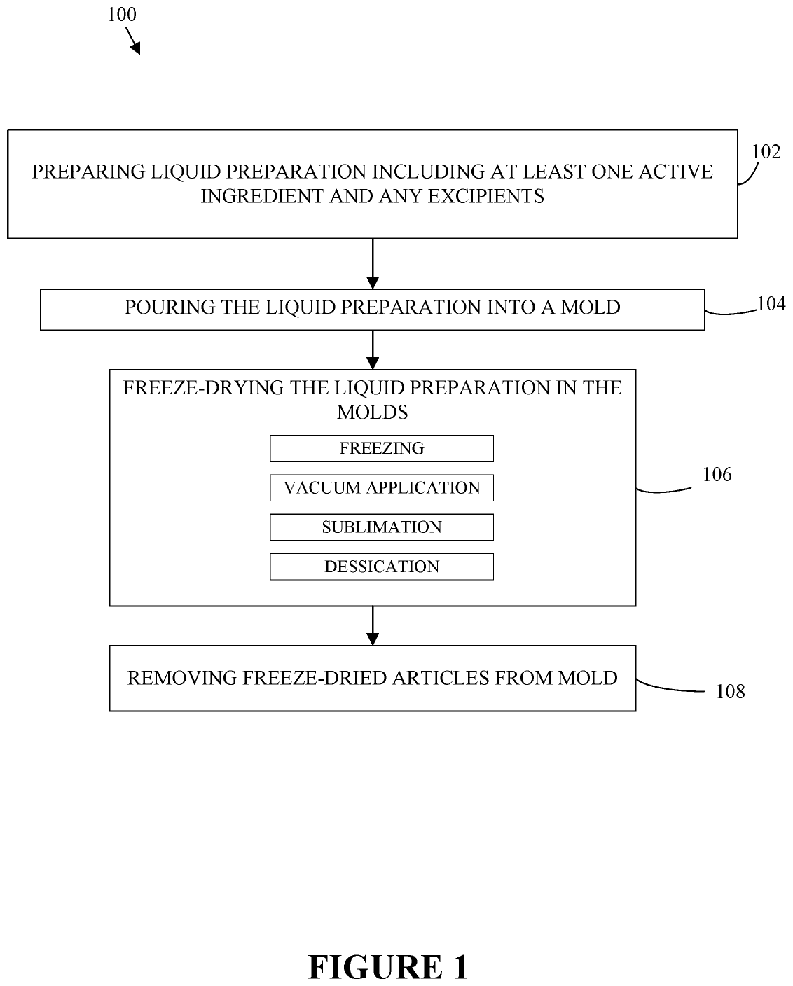

FIG. 1 is a flow diagram illustration and example embodiment of an exemplary method 100 for making the rapidly dissolving articles including the above formulations for use as skincare tablets. At 102, the method comprises combining a desired amount of the at least one active ingredients as listed above and forming an aqueous solution or suspension. In some embodiments, the formula may include one or more of the listed excipients in addition to the active ingredients. Determining the desired amount of each ingredient to be combined may include weighing/measuring the ingredients. In some embodiments, the weighing of ingredients (e.g., the at least one active ingredient) provides for a cosmetic formula that is defined by the weight percent of its constituents. The active ingredients and the excipients may be combined in a vessel or a processing tank with sterile water to form the liquid preparation. The active ingredients and excipients may be added individually and agitated/stirred, with a magnetic stir plate and bar for instance, until completely dissolved in the sterile water. The liquid preparation will be completely formed once all of the active and inactive ingredients have been added to the desired weight percent. The liquid preparation may be recirculated and mixed for additional time to ensure proper dissolution of the ingredients.

At 104, in one embodiment of the method the next step may comprise pouring the liquid preparation into a mold. The mold may be a tray with a plurality of rows and a plurality of columns of individual cells configured to receive the liquid preparation. The liquid preparation may be pumped and dispensed into the cells using an automatic or robotic process. It will be appreciated that the liquid preparation may also be dispensed into the cells manually. During the dispensing process, continuous monitoring and calibration may occur to ensure that each cell is being filled with a target volume and therefore weight of the liquid preparation. It will be appreciated that this target weight may vary depending on the ingredients in the formula and the amount of the ingredients included for one dose of the cosmetic or pharmaceutical.

At 106, in one embodiment of the method, the next step of the method is freeze-drying the liquid preparation. The trays may be inserted into the freeze dryer to begin a freeze-drying process. A plurality of trays may be placed into the freeze dryer to go through the freeze-drying process simultaneously. Once the trays are loaded into the freeze dryer, the freeze-drying process may include a freezing step, a vacuum step, a primary drying or sublimation step, and a secondary drying or desiccation step. The freezing and drying steps may include a ramp step where the temperature and/or pressure is decreased or increased over a set time interval and a hold step where the temperature and/or pressure in the freeze dryer is maintained over a set time interval.

An exemplary freeze-drying process is illustrated in the table below. It will be appreciated that the parameters like the target temperatures, target pressures, ramp time intervals, and holding time intervals may all vary depending on the formula in the liquid preparation and best practices. Once the second drying sub-steps have been completed, the trays made be stored in the freeze dryer where the temperature is controlled or unloaded immediately.

| | | Min | | Max | |
|---|---|---|---|---|---|
| Steps | Parameters | Range | | Range | |
| Loading | Freezer Temperature | −50° | C. | +25° | C. |
| | for loading | | | | |
| Freezing | Freezer temperature | −50° | C. | +5° | C. |
| | Ramp Time Interval | 0 | min | 1200 | min |
| | Holding Time Interval | 120 | min | 1440 | min |
| Vaccuum | Freezer Pressure | 10 | μbar | 600 | μbar |
| Sublimation/ | Freezer temperature | −50° | C. | −10° | C. |
| Primary drying | Ramp Time Interval | 0 | min | 2400 | min |
| | Holding Time Interval | 30 | min | 10080 | min |
| | Freezer Vacuum Pressure | 10 | μbar | 600 | μbar |
| Dessiccation/ | Freezer Temperature | +15° | C. | +40° | C. |
| Secondary | Freezer Vacuum Pressure | 10 | μbar | 600 | μbar |
| drying | Ramp Time Interval | 0 | min | 1200 | min |
| | Holding Time Interval | 120 | min | 5760 | min |

Alternatively, at 106, the freeze-drying process may include a plurality of freezing sub-steps, followed by a plurality of sublimation or primary drying sub-steps, and followed by a plurality of desiccation or secondary drying sub-steps. In each sub-step, the freeze dryer may vary the temperature and the pressure. The sub-steps may include a ramp step where the temperature is decreased or increased over a set time interval and a hold step where the temperature in the freeze dryer is maintained over a set time interval.

An exemplary freeze-drying process is illustrated in the table below. It will be appreciated that the freeze-drying process may include more or less freezing sub-steps and more or less primary and second drying sub-steps. Further, the parameters like the target temperatures, target pressures, ramp time intervals, and holding time intervals may all vary depending on the formula in the liquid preparation and best practices. Once the second drying sub-steps have been completed, the trays made be stored in the freeze dryer where the temperature is controlled or unloaded immediately.

| Step | Sub-step | Parameters | |
|---|---|---|---|
| Freezing | Starting Temperature | 5° C. ± 2° C. | |
| | Holding Time Interval | ≥60 | min |
| Freezing | Sub-step Target Temperature | 0° C. ± 2° C. | |
| | Ramp Time Interval | ≥30 | min |
| | Holding Time Interval | ≥60 | min |
| Freezing | Sub-step Target Temperature | −5° C. ± 2° C. | |
| | Ramp Time Interval | ≥30 | min |
| | Holding Time Interval | ≥60 | min |
| Freezing | Sub-step Target Temperature | −10° C. ± 2° C. | |
| | Ramp Time Interval | ≥30 | min |
| | Holding Time Interval | ≥60 | min |
| Freezing | Sub-step Target Temperature | −15° C. ± 2° C. | |
| | Ramp Time Interval | ≥30 | min |
| | Holding Time Interval | ≥60 | min |
| Freezing | Sub-step Target Temperature | −50° C. ± 2° C. | |
| | Ramp Time Interval | ≥60 | min |
| | Holding Time Interval | ≥240 | min |
| Vaccuum | Target Pressure | 35 ± 50 | μbar |
| Application | Ramp Time Interval | ≤60 | min |
| Sublimation/ | Sub-step Target Temperature | −45° C. ± 2° C. | |
| Primary drying | Ramp Time Interval | ≥120 | min |
| | Holding Time Interval | ≥4360 | min |
| | Vacuum Pressure | 35 ± 50 | μbar |
| Sublimation/ | Sub-step Target Temperature | −35° C. ± 2° C. | |
| Primary drying | Ramp Time Interval | ≥150 | min |
| | Holding Time Interval | ≥860 | min |
| | Vacuum Pressure | 35 ± 50 | μbar |
| Sublimation/ | Sub-step Target Temperature | −10° C. ± 2° C. | |
| Primary drying | Ramp Time Interval | ≥30 | min |
| | Holding Time Interval | ≥240 | min |
| | Vacuum Pressure | 35 ± 50 | μbar |
| Sublimation/ | Sub-step Target Temperature | 25° C. ± 2° C. | |
| Primary drying | Ramp Time Interval | ≥360 | min |
| | Vacuum Pressure | 35 ± 50 | μbar |
| Desiccation/ | Sub-step Temperature | 25° C. ± 2° C. | |
| Secondary | Vacuum Pressure | 35 ± 50 | μbar |
| drying | Holding Time Interval | ≥1125 | min |
| Storage (if not | Sub-step Temperature | −20° C. ± 2° C. | |
| unloading) | Ramp Time Interval | ≥10 | min |
| | Holding Time Interval | ≥2400 | min |
| Unloading | Freezer Temperature | ≥20° | C. |
| | Tablet Temperature | ≥15° | C. |

At 108, in one embodiment of the method, the next step comprises removing the rapidly dissolving articles from the trays. The trays may be removed from the freeze dryer and the rapidly dissolving articles may be removed from the tray one tray at a time. The rapidly dissolving articles may be removed from the trays manually or by some automated process.

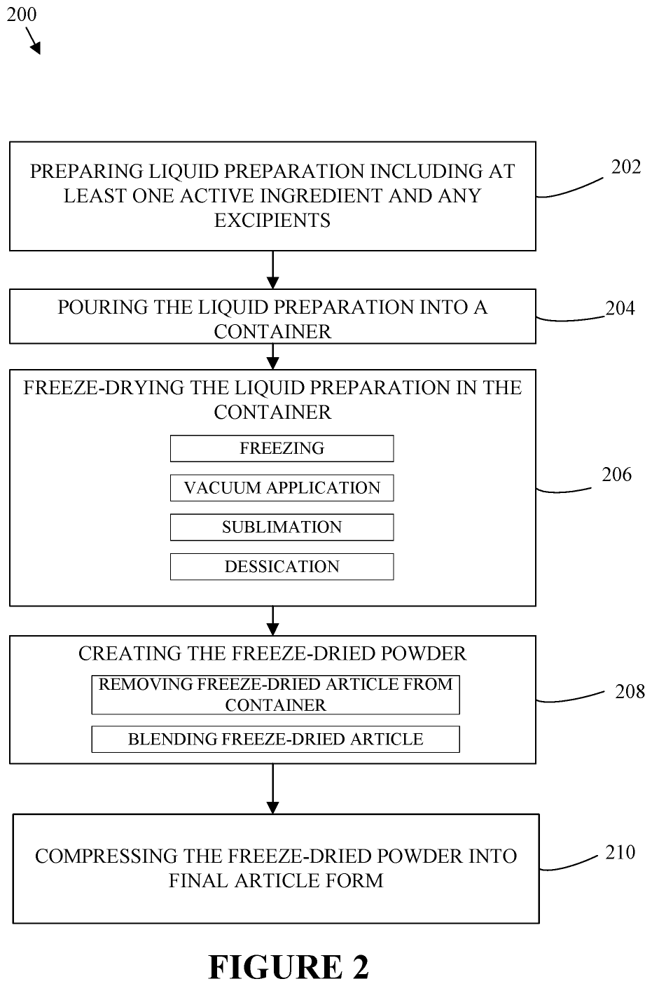

FIG. 2 is a flow diagram illustration and example embodiment of a second exemplary method 200 for making the rapidly dissolving articles including the above formulations for use as skincare tablets. At 202, the method comprises combining a desired amount of the at least one active ingredients as listed above and forming an aqueous solution or suspension. This process is substantially the same as 102 described above.

At 204, the second exemplary method may include pouring the liquid preparation into a container. The liquid preparation may be poured into one larger container as opposed to being dispensed into individual cells.

At 206, the second exemplary method may include freeze drying the liquid preparation in the container to obtain a freeze-dried article. The freeze-drying process may be substantially the same as 106 described above. It will be appreciated that the number of freezing and drying steps as well as the freezing and drying target temperatures, pressure, and time parameters may be altered to freeze dry all of the liquid preparation in one batch.

At 208, the second exemplary method may comprise creating a freeze-dried powder from the freeze-dried article in the container. The freeze-dried article may be removed from the container. The freeze-dried article may be blended in to a free-dried powder. It will be appreciated that the freeze-dried article may be altered to a freeze-dried powder through another process like crushing or grinding.

At 210, the second exemplary method may include compressing the freeze-dried powder into a final article form. The freeze-dried powder can be placed into a mold and pressed to produce the final article form. For instance, the powder may be pressed into a mono-dosed tablet of the topical cosmetic or pharmaceutical.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. An article for use as a cosmetic or pharmaceutical, comprising:
a molded, dried preparation comprising greater than zero and up to fifty percent water by weight, the dried preparation formed by drying a liquid preparation, the liquid preparation comprising:
a base material comprising one or more of: alginate, chitosan, collagen, sodium hyaluronate, and trehalose;
an active ingredient comprising one or more of: amino acids, enzymes and coenzymes, sugars, saccharides, glycols, polyols, nutrients, phenols, plant extracts, peptides, proteins, ultraviolet (UV) filters, vitamins, organic compounds, inorganic compounds, antioxidants, prebiotics, probiotics, postbiotics, fatty acids, glycerol, triglycerides, monoglycerides, diglycerides, phospholipids, steroids, sterols, sphingolipids, alpha hydroxy acid (AHA), a beta hydroxy acid (BHA), or a polyhydroxy acid (PHA), and oils; and
an inactive ingredient, comprising one or more of: amino acids, alcohols, sugars, saccharides, polyols, glycols, polymers, acids, esters, solvents, proteins, surfactants, buffers, chelating complexes, and inorganic salts;
wherein the dried preparation is configured to be rehydrated with an aqueous based solvent, solution or emulsion to form a ready-to-use liquid or semi-solid topical product.

2. The article of claim 1, wherein the base material of the liquid preparation comprises one of more of alginate, chitosan, sodium hyaluronate, or collagen, and an active ingredient comprises alpha arbutin, wherein the dried preparation comprises 5-15% water by weight, 50-70% active ingredient by weight, and 15-40% base material by weight.

3. The article of claim 1, wherein the base material of the liquid preparation comprises one of more of alginate, chitosan, sodium hyaluronate, or collagen, and an active ingredient comprises potassium azeloyl diglycinate, wherein the dried preparation comprises 5-15% water by weight, 60-80% active ingredient by weight, and 10-30% base material by weight.

4. The article of claim 1, wherein the base material of the liquid preparation comprises one of more of alginate, chitosan, sodium hyaluronate, or collagen, and an active ingredient comprises potassium niacinamide USP, wherein the dried preparation comprises 5-15% water by weight, 50-70% active ingredient by weight, and 15-40% base material by weight.

5. The article of claim 1, wherein the base material of the liquid preparation comprises one of more of alginate, chitosan, sodium hyaluronate, or collagen, and active ingredients comprising gluconolactone USP, dextran, maltobionic acid, lactobionic acid, and aloe barbadensis leaf juice, wherein the dried preparation comprises 5-15% water by weight, 70-90% active ingredients by weight, and 5-20% base material by weight.

6. The article of claim 1, wherein the base material of the liquid preparation comprises one of more of alginate, chitosan, sodium hyaluronate, or collagen, and active ingredients comprising tripeptide-1 (and) copper peptide 1, acetyl hexapeptide-8, palmitoyl tripeptide-1, and palmitoyl tetrapeptide-7, wherein the dried preparation comprises 5-15% water by weight, 30-50% active ingredients by weight, and 40-60% base and/or inactive ingredient material by weight.

7. The article of claim 1, wherein the base material of the liquid preparation comprises one of more of alginate, chitosan, sodium hyaluronate, or collagen, and active ingredients comprising hydroxypinacolone retinoate, and DL-alpha tocopheryl acetate, wherein the dried preparation comprises 10-30% water by weight, 50-70% active ingredients by weight, 1-10% base material by weight; and 10-20% dextran by weight.

8. The article of claim 1, comprising sodium hyaluronate, betaine, polyglutamic acid as active ingredients, and either alginate, chitosan, sodium hyaluronate, or collagen as a base.

9. The article of claim 1, comprising Centella asiatica Extract, Camellia sinensis extract, and either alginate, chitosan, sodium hyaluronate, or collagen as a base.

10. The article of claim 1, comprising glycolic acid, lactic acid, phytic acid, mandelic acid, salicylic acid, and either alginate, chitosan, sodium hyaluronate, or collagen as a base.

11. The article of claim 1, comprising l-ascorbic acid, DL-alpha tocopheryl acetate, ferulic acid, and propanediol or ethoxydiglycol, and either alginate, chitosan, sodium hyaluronate, or collagen as a base.

12. A method for making a dried and molded article that is configured to be rehydrated for use as a cosmetic or pharmaceutical, the method comprising
combining at least one active ingredient from a selection of active ingredients with an aqueous based solvent, to form an active aqueous solution or suspension, wherein the selection of active ingredients comprises: amino acids, enzymes and coenzymes, sugars, saccharides, glycols, polyols, nutrients, phenols, plant extracts, peptides, proteins, ultraviolet (UV) filters, vitamins, organic compounds, inorganic compounds, antioxidants, prebiotics, probiotics, postbiotics, fatty acids, glycerol, triglycerides, monoglycerides, diglycerides, phospholipids, steroids, sterols, sphingolipids, alpha hydroxy acid (AHA), a beta hydroxy acid (BHA), or a polyhydroxy acid (PHA), and oils;
combining a base material with the active aqueous solution or suspension, wherein the base material comprises one or more of: alginate, chitosan, collagen, sodium hyaluronate, and trehalose;
combining at least one inactive ingredient with the active aqueous solution or suspension to form a liquid preparation, wherein the inactive ingredient comprises one or more of: amino acids, alcohols, sugars, saccharides, polyols, glycols, polymers, acids, esters, solvents, proteins, surfactants, buffers, chelating complexes, and inorganic salts; and
creating a molded and dried preparation comprising greater than zero and up to forty percent water by weight, wherein the dried preparation created by drying the liquid preparation;
wherein the dried preparation is configured to be rehydrated with an aqueous based solvent, solution or emulsion to form a ready-to-use liquid or semi-solid topical product.

13. The method of claim 12, wherein creating the molded and dried preparation comprises freeze-drying the liquid preparation.

14. The method of claim 12, wherein the base material of the liquid preparation comprises one of more of alginate, chitosan, sodium hyaluronate, or collagen, and an active ingredient comprises alpha arbutin, wherein the dried preparation comprises 5-15% water by weight, 50-70% active ingredient by weight, and 15-40% base material by weight.

15. The method of claim 12, wherein the base material of the liquid preparation comprises one of more of alginate, chitosan, sodium hyaluronate, or collagen, and an active ingredient comprises potassium azeloyl diglycinate, wherein the dried preparation comprises 5-15% water by weight, 60-80% active ingredient by weight, and 10-30% base material by weight.

16. The method of claim 12, wherein the base material of the liquid preparation comprises one of more of alginate, chitosan, sodium hyaluronate, or collagen, and an active ingredient comprises potassium niacinamide USP, wherein the dried preparation comprises 5-15% water by weight, 50-70% active ingredient by weight, and 15-40% base material by weight.

17. The method of claim 12, wherein the base material of the liquid preparation comprises one of more of alginate, chitosan, sodium hyaluronate, or collagen, and active ingredients comprising gluconolactone USP, dextran, maltobionic acid, lactobionic acid, and aloe barbadensis leaf juice, wherein the dried preparation comprises 5-15% water by weight, 70-90% active ingredients by weight, and 5-20% base material by weight.

18. The method of claim 12, wherein the base material of the liquid preparation comprises one of more of alginate, chitosan, sodium hyaluronate, or collagen, and active ingredients comprising tripeptide-1 (and) copper peptide 1, acetyl hexapeptide-8, palmitoyl tripeptide-1, and palmitoyl tetrapeptide-7, wherein the dried preparation comprises 5-15% water by weight, 30-50% active ingredients by weight, and 40-60% base and/or inactive ingredient material by weight.

19. The method of claim 12, wherein the base material of the liquid preparation comprises one of more of alginate, chitosan, sodium hyaluronate, or collagen, and active ingredients comprising hydroxypinacolone retinoate, and DL-alpha tocopheryl acetate, wherein the dried preparation comprises 10-30% water by weight, 50-70% active ingredients by weight, 1-10% base material by weight; and 10-20% dextran by weight.

20. A preparation for use as a topical cosmetic or pharmaceutical, comprising:
a base material comprising one or more of: alginate, chitosan, collagen, sodium hyaluronate, and trehalose;
an active ingredient comprising one or more of: amino acids, enzymes and coenzymes, sugars, saccharides, glycols, polyols, nutrients, phenols, plant extracts, peptides, proteins, ultraviolet (UV) filters, vitamins, organic compounds, inorganic compounds, antioxidants, prebiotics, probiotics, postbiotics, fatty acids, glycerol, triglycerides, monoglycerides, diglycerides, phospholipids, steroids, sterols, sphingolipids, alpha hydroxy acid (AHA), a beta hydroxy acid (BHA), or a polyhydroxy acid (PHA), and oils; and
an inactive ingredient, comprising one or more of: amino acids, alcohols, sugars, saccharides, polyols, glycols, polymers, acids, esters, solvents, proteins, surfactants, buffers, chelating complexes, and inorganic salts;
wherein the base material, active ingredient, and inactive ingredient are prepared as a liquid preparation;
wherein the liquid preparation is freeze dried to form a molded, dried preparation comprising greater than zero and up to thirty percent water by weight; and
wherein the dried preparation is configured to be rehydrated with an aqueous based solvent, solution or emulsion to form a ready-to-use liquid or semi-solid topical preparation.
